(19) United States
(12) Patent Application Publication
(10) Pub. No.: US 2006/0019954 A1
(43) Pub. Date: Jan. 26, 2006

(54) Method for reducing the likelihood of the occurrence of cardiac arrhythmias

(75) Inventor: Peng-Sheng Chen, La Canada, CA (US)

Correspondence Address: JONES DAY, 555 SOUTH FLOWER STREET FIFTIETH FLOOR, LOS ANGELES, CA, 90071, US

(73) Assignee: Cedars-Sinai Medical Center

(21) Appl. No.: 10/895,013

(22) Filed: Jul. 20, 2004

(51) Int. Cl. A61K 31/537

(52) U.S. Cl. 514/237.5

(57) ABSTRACT

A method is described for reducing the likelihood of the occurrence of a cardiac arrhythmias of the type leading to sudden cardiac death in a mammalian subject. The method comprises administering to the mammalian subject an effective amount of a composition to agonize β₃ adrenergic receptors in the mammalian subject. The composition may comprise one or more agents that are known to agonize β₃ adrenergic receptors, such as BRL 37344, CPG 12177, CL 316243, SR 58661, pindolol, and cyanopindolol.

### GOVERNMENT INTEREST

This invention was made in part with government support under Grant R01 HL66389, awarded by the National Institutes of Health. The government has certain rights to this invention.

### FIELD OF THE INVENTION

The invention generally relates to a method for reducing the likelihood of the occurrence of cardiac arrhythmias of the type that can result in sudden cardiac death.

### BACKGROUND OF THE INVENTION

Sudden cardiac death (SCD) is a major public health problem that accounts for more than half of all cardiovascular deaths. SCD takes the lives of approximately 450,000 people in the United States each year, more than lung cancer, breast cancer, stroke, and AIDS combined. Most cases of SCD are due to ventricular arrhythmias and there is often an element of underlying ischemic heart disease. Ventricular tachycardia (VT) and ventricular fibrillation (VF) are different types of ventricular arrhythmias. VT is an abnormally fast ventricular heart rhythm which is, by itself, typically not fatal. VF is a chaotic ventricular heart rhythm which produces little or no net blood flow from the heart, such that there is little or not net blood flow to the brain and other organs. VF, if not terminated, results in death. Patient groups most at risk of ventricular arrhythmias leading to SCD include those with an acute or chronic myocardial infarction. Accordingly, deaths from SCDs may be lowered by preventing the specific heart rhythm disturbances (ventricular arrhythmias) associated with it.

Different treatment options exist for SCD. The most common treatment includes implantable cardiac defibrillators (ICD) and drug therapy. ICDs have been available in the United States since the mid-1980s and have a well-documented success rate in decreasing the rate of death of patients at high risk for SCD. A major trial conducted by the U.S. National Institutes of Health (the Anti-arrhythmics Versus Implantable Defibrillator or AVID trial) compared therapy with the best available anti-arrhythmic drugs with ICD therapy for patients with spontaneous ventricular tachycardia or ventricular fibrillation. The overall death rate in the ICD patient group was found to be 39% lower than the death rate of patients treated with anti-arrhythmic drugs after only 18 months mean follow-up.

However, ICD therapy has the disadvantages of being surgically invasive and expensive to implant and maintain over the lifetime of a patient. Complications from the insertion of ICDs are common, such as those relating to lead dislodgment, bleeding problems and erosions, and occasionally more serious occurrences such as thromboembolism or infection. Inappropriate shocks are also common and can significantly impinge on the patient's quality of life. Another complication associated with the ICD is its paradoxical ability to provoke arrhythmia in certain cases.

Accordingly, anti-arrhythmic drugs present a desirable alternative or a concomitant therapy to ICD implantation. Anti-arrhythmic drugs are commonly divided into four classes according to their electrophysical mode of action. Class I anti-arrhythmic drugs (sodium channel blockers) work by blocking the sodium channels in order to slow impulse conduction in the heart. Class II anti-arrhythmic drugs (β-blockers) slow the heart rate and force contraction by decreasing the sensitivity of cells to adrenaline and adrenaline-like substances that act at β receptors. Class III anti-arrhythmic drugs (potassium channel blockers), such as amiodarone and solatol, work, in part, by prolonging the recovery time of cardiac cells after they have carried an impulse. This can prevent the electrical pathways of the heart from causing an arrhythmia or permit only slower arrhythmias. Lastly, Class IV (calcium channel blockers), such as verapamil and diltiazem, slow the heart rate by blocking the heart cells' calcium channels and therefore slowing the conduction at the AV node.

Many of the anti-arrhythmic drugs, however, have been associated with severe and sometimes life-threatening side effects. For example, certain Class I anti-arrhythmic drugs play a minimal role in improving overall survival for patients at risk for SCD. Indeed, studies have suggested that certain of Class I drugs may be pro-arrhythmic and associated with an increase in overall mortality. In fact, certain Class I drugs have been associated with a 21% increase in death rates among people at risk for SCD.

Amiodarone, a Class III anti-arrhythmic drug, has been identified as among the most effective of anti-arrhythmic drugs, decreasing mortality by 13 to 19%. Still, amiodarone is limited by the possibility of serious and even fatal side effects such as lung problems, liver problems, and new or worsening irregular heartbeats. Lung toxicity caused by amiodarone can be fatal, and once lung toxicity is diagnosed, the best option is to stop the taking the drug. However, the toxic effect of amiodarone often persists because of its long half-life of up to 45 days.

Despite advances in the prevention and treatment of heart disease and improvements in emergency transport, the risk of SCD among post-myocardial infarction patients remains high. The use of anti-arrhythmic drugs, alone or in connection with ICD therapy, has and will continue to have a significant role in decreasing the incidence of SCD. Unfortunately, many anti-arrhythmic drugs present a risk of serious side effects on patients and can, in some instances, be pro-arrhythmic or even fatal at both therapeutic and toxic drug concentrations.

### SUMMARY OF INVENTION

Cardiac myocytes are known to express at least three types of β adrenergic receptors (β₁, β₂ and β₃) and activation of these receptors may have significant effects on heart rhythm. The effects of β₁ and β₂ adrenergic receptors are well-established in both human and other mammals. Stimulation of β₁ and β₂ adrenergic receptors is pro-arrhythmic while the blockage of these receptors is anti-arrhythmic. The effect of β₃ adrenergic receptors on heart rhythm, however, has not been clearly understood. The methods disclosed herein are based on the finding that β₃ adrenergic receptor stimulation is anti-arrhythmic.

Accordingly, methods are provided for reducing the likelihood of the occurrence of a cardiac arrhythmia in a mammalian subject comprising administering to the mammalian subject an effective amount of a composition to agonize β₃ adrenergic receptors.

Compositions that agonize β₃ adrenergic receptors may comprise one or more pharmaceutical agents known to agonize β₃ adrenergic receptors and which are anti-arrhythmic. In addition, the composition may further comprise additional pharmaceutical agents that are known to have an anti-arrhythmic effect, such as sodium channel blockers, β₁ and β₂ blockers, potassium channel blockers, and calcium channel blockers, to name a few.

Compositions suitable for the practice of the instant method comprise agents that agonize β₃ adrenergic receptors. The agents may, in addition to agonizing β₃ adrenergic receptors, antagonize β₁ and/or β₂ adrenergic receptors. Suitable agents used in the composition may include BRL 37344, CPG 12177, CL 316243, SR 58661, pindolol, and cyanopindolol. The composition may employ liquid or solid form pharmaceutical preparations in combination with a pharmaceutically acceptable carrier.

Additional agents that agonize β₃ adrenergic receptors may readily be determined by standard and well-known procedures, such as evaluating the affinity of a suspected agent for the different beta adrenergic receptor subtypes (β₁, β₂ and β₃) and comparing the activity of the suspected agent with the various receptor subtypes. Because the effects of agonizing β₁ and β₂ adrenergic receptors are pro-arrhythmic, suitable agents should selectively agonize β₃ adrenergic receptors.

The effective amount of a composition that is capable of agonizing β₃ adrenergic receptors in a mammalian subject depends upon the age, state of health, the weight of the subject, the extent of the disease, and the frequency and route of administration.

Conventional techniques for studying arrhythmias may be employed to determine the effectiveness of a specific dose or amount of a composition to be used in treating arrhythmias. These techniques include ambulatory electrocardiography with computer-assisted analysis and programmed stimulation techniques for arrhythmia induction during intracardiac electrophysiological study, to name a few. Other standard and well-known techniques may also be used in determining the effective amount of a particular composition to agonize β₃ adrenergic receptors.

The administration of compositions containing β₃ adrenergic receptor agonists may be accomplished by a variety of routes known to those of ordinary skill in the art, including but not limited to oral, parenteral, transdermal, transmyocardial, and rectal administration, to name a few.

The above and other objects, features and advantages will become apparent to those skilled in the art from the following description of the preferred embodiments.

### BRIEF DESCRIPTION OF THE DRAWINGS

FIG. 1 depicts stained tissue specimens from the left ventricle around the posterior and anterior papillary muscles obtained from the canine SCD Group. The tissue specimens were stained by a modified immunohistochemical ABC method with primary antibodies against β₃ adrenergic receptors in FIGS. 1A-B and with primary antibodies against β₃ adrenergic receptors in FIGS. 1C-E. A specimen with negative control staining without the primary antibodies is depicted in FIG. 1F. (FIGS. 1A-B and 1D under 20× magnification and FIGS. 1C, 1E and 1F under 40× magnification).

FIG. 2 is a high power view of stained tissue specimens from the left ventricle around the posterior and anterior papillary muscles obtained from the canine SCD Group showing distribution of the β₁ and β₃ adrenergic receptors. FIG. 2A depicts the β₃ adrenergic receptor distribution and FIG. 2B depicts the β₁ adrenergic receptor distribution. (FIGS. 2A-B under 100× magnification).

FIG. 3 are confocal microscope images of double-immunofluorescence stained tissue specimens from the left ventricle obtained from the canine SCD Group showing the partial co-localization of the β₁ and β₃ adrenergic receptors. FIG. 3A shows the β₃ adrenergic receptor positive sarcolemma and Z bands within the cardiac myocytes; FIG. 3B shows the β₁ adrenergic receptor positive sarcolemma and Z bands of cardiac myocytes; FIG. 3C shows the merged view of the images in FIGS. 3A and 3B which represents the same single confocal optical plane; and FIG. 3D shows the image analysis of the cross-section of the merged maximum intensity projection of FIG. 3C along the XZ and YZ planes of the three-dimensional volume of a stack of optical sections from a z-series. (FIGS. 3A-D under 40× magnification).

FIG. 4 shows β₃ adrenergic receptor immunostained tissue specimens from the left ventricular free wall obtained from four canine groups, with the positive staining: the Control Group (FIG. 4A); the MI Group (FIG. 4B); the RSG Group (FIG. 4C); and the SCD Group (FIG. 4D). (FIGS. 4A-D under 40× magnification).

FIG. 5 depicts examples of β₃ adrenergic receptor immunostained tissue samples from the left ventricular free wall obtained from all twenty-seven (27) canine subjects from the four canine groups. FIG. 5A shows the canine subjects 1 through 6 (left to right) from the Control Group; FIG. 5B shows the canine subjects 1 through 6 from the MI Group; FIG. 5C shows the canine subjects 1 through 6 from the RSG Group; and FIG. 5D shows canine subjects 1 through 9 from the SCD Group. (FIGS. 5A-D under 20× magnification).

FIG. 6 shows representative Western blotting bands of β₃ adrenergic receptor, β₁ adrenergic receptor, β₂ adrenergic receptor, NGF, TH and GAPD obtained from fresh tissue samples from the non-infarcted left ventricular free wall of the Control Group (FIG. 6A) and in the SCD Group (FIG. 6B).

FIG. 7 are graphs showing the β adrenergic receptor protein and mRNA levels in the fresh tissue samples from the non-infarcted left ventricular free wall of the Control Group (hatched column) and SCD Group (filled column). FIG. 7A shows the β adrenergic receptor protein expression level and FIG. 7B is a graph showing the β adrenergic receptor mRNA expression levels. The data in FIGS. 7A-B were expressed as the ratio of β adrenergic receptors to GAPDH.

FIG. 8 depicts ECG recordings showing two short runs of VT episodes in a SCD canine subject receiving subcutaneous infusion of BRL 37344 (2 μg/kg/hr) via osmotic pump.

FIG. 9A shows the occurrence of phase 2 VT episodes and FIG. 9B is a graph showing the QTc intervals of the two SCD canine subject receiving subcutaneous infusion of BRL 37344 (2 μg/kg/hr) via osmotic pump throughout follow-up.

### DESCRIPTION OF THE PREFERRED EMBODIMENTS

β₃ adrenergic receptor stimulation has been demonstrated to reduce the occurrence of ventricular arrhythmia and SCD in canine SCD models. The unique profiles of the various β adrenergic receptors subtypes make it possible to selectively stimulate or inhibit β₃ adrenergic receptors with little effects on either β₁ or β₂ adrenergic receptors. β₃ adrenergic receptors differ significantly from β₁ and β₂ adrenergic receptors. The β₃ adrenergic receptor is a G-protein-coupled seven-transmembrane domain receptor that interacts with either Gs or Gi proteins. β₃ adrenergic receptors lack phosphorylation sites for PKA or β-ARK, so it is relatively resistant to desensitization. The β₃ adrenergic receptor is also resistant to long term down regulation and is activated at higher concentrations of catecholamines than β₁ and β₂ adrenergic receptors.

Accordingly methods are provided for reducing the likelihood of the occurrence of a cardiac arrhythmia comprising administering an effective amount of a composition to agonize β₃ adrenergic receptors in a heart of a mammalian subject susceptible to having an arrhythmia.

The unique pharmacological profile of β₃ adrenergic receptors make it possible to stimulate or inhibit β₃ adrenergic receptor with little effects on β₁ and/or β₂ adrenergic receptors. For example, β₃ adrenergic receptors are potently activated by agonists that weakly interact with β₁ and β₂ adrenergic receptors, such as BRL 37344, CL 316243 and SR 58611 and β₃ adrenergic receptors are blocked by either selective β₃ adrenergic receptor antagonists, such as SR 59230 or β₁ , β₂ and β₃ adrenergic receptor antagonists, such as bupranolol. β₃ adrenergic receptors also undergo partial activation in response to several β₁ and β₂ adrenergic receptor antagonists, such as CGP 12177 pindolol and cyanopindolol and are weakly blocked by conventional β₁ and β₂ antagonists, such as propranolol and nadolol.

Compositions suitable in the practice of the instant methods include agents that agonize β₃ adrenergic receptors and include, by way of example, but are not limited to: BRL 37344, CPG 12177, CL 316243, SR 58661, pindolol, and cyanopindolol. Suitable agents may also have the dual therapeutic property of acting both as a β₃ adrenergic receptor agonist and as β₁ and β₂ blockers.

Additional agents suitable for practicing the methods described herein may also be identified by standard and well-known procedures by which the specificity and agonistic activity of a particular agent for β₃ adrenergic receptors may be determined.

The agonistic activity of a particular agent for β₃ adrenergic receptors may be evaluated by testing the capacity of a agent to produce dose-dependent increases in intracellular cAMP concentrations in comparison to known β₃ adrenergic receptor agonists. This can be determined by standard procedures well-known to one of skill in the art and are disclosed in U.S. Pat. No. 6,469,031, which is herein incorporated by reference.

For example, the extent to which a agent agonizes β₃ adrenergic receptors may be determined by exposing the agent to cells expressing β₃ adrenergic receptors and by determining the dose-dependent increases in intracellular cAMP concentration. An estimate of the cyclase stimulation constant may be derived for each agent and the intrinsic activity of the suspected agonistic agent may be evaluated. The intrinsic activity may be defined as the ratio between the maximal effect of an agonist and the maximal effect produced by known β₃ agonists. The specificity of agents as agonists for β₃ adrenergic receptors may also be readily determined by evaluating the affinity of the agent for β₁ and β₂ adrenergic receptors.

β₃ adrenergic receptor agonists may have other indications for the treatment of β₃ adrenergic receptor mediated conditions, such as diabetes, obesity, gastrointestinal disorders including irritable bowel syndrome and intestinal hypermotility disorders, including peptic ulcerations, esophagitis, gastritis, duodenities, intestinal ulcerations including inflammatory bowel disease, ulcerative colitis, Crohn's disease and proctitis, and gastrointestinal ulcerations, as well as neurogenetic inflammation such as cough and asthma, and depression.

Accordingly, the methods disclosed herein may provide treatment of other β₃ adrenergic receptor mediated conditions. For example, agonists selective for β₃ adrenergic receptors are known to be useful in the treatment of obesity and diabetes in mammals, as well as in the treatment of gastrointestinal disorders and neurogenetic inflammation. Additionally, they are known to lower triglyceride and cholesterol levels and to raise high density lipoprotein levels.

The compositions that agonize β₃ adrenergic receptor may also contain other conventional pharmaceutically acceptable compounding ingredients, generally referred to as carriers or diluents, as necessary or desired. In addition, the compositions may be preserved by the addition of an antioxidant such as ascorbic acid or by other suitable preservatives. Conventional procedures for preparing such compositions in appropriate dosage forms can be utilized.

The composition may contain a single agent or a combination of pharmaceutical agents. For example, the agents may be combined with other known anti-arrhythmic compounds, such as β₁ and β₂ adrenergic receptor antagonists.

Administration of the compositions can be carried out orally or parenterally employing liquid or solid form pharmaceutical preparations containing β₃ adrenergic receptor agonists in combination with a pharmaceutically acceptable carrier.

Compositions containing β₃ agonizing agents may be administered through different modes of entry into the mammalian subject, including oral, parental, transdermal, transmyocardial, and rectal administration, to name a few.

For oral administration, the composition may be formulated into solid or liquid preparations, such as capsules, pills, tablets, troches, lozenges, melts, powders, solutions, suspensions, or emulsions, and may be prepared according to methods known to the art for the manufacture of pharmaceutical compositions. The solid unit dosage forms may be a hard or soft shelled gelatin capsule containing, for example, surfactants, lubricants, and inert fillers such as lactose, sucrose, calcium phosphate, and corn starch.

The composition may also be administered parenterally as injectable dosages in a physiologically acceptable diluent with a pharmaceutical carrier. Parental administration may be subcutaneous, intravenous, intramuscular, or interperitoneally.

The composition may also be administered by transdermal delivery devices or patches. Such transdermal delivery devices may be used to provide continuous or discontinuous infusion of the compositions in controlled amounts. The construction and use of transdermal patches for the delivery of pharmaceutical agents are well known in the art and disclosed in U.S. Pat. No. 5,023,252, which is herein incorporated by reference. Such patches may be constructed for continuous, pulsatile, or on demand delivery of pharmaceutical agents.

The composition may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

It may be desirable or necessary to introduce the composition to the patient by a mechanical delivery device. The construction and use of mechanical delivery devices for the delivery of pharmaceutical agents is well known in the art. One such implantable delivery system, used for the transport of agents to specific anatomical regions of the body, is described in U.S. Pat. No. 5,011,472 which is herein incorporated by reference.

The effective amount of a composition is the amount or dosage sufficient to agonize β₃ adrenergic receptors and to have an anti-arrhythmic effect. The amount of the composition to be administered depends on considerations such as the particular compound and dosage unit employed, the mode of administration, the period of treatment, the age, sex and weight of the patient treated, and the nature and extent of the condition treated. The effective amount can readily be determined based upon standard laboratory techniques known to evaluate compound receptor site inhibition, by standard toxicity tests and by standard pharmacological assays.

For example, intravenous administration of 2 μg/kg/hr of BRL 37344 by an osmotic pump for a two week period in mammalian SCD models has been shown to be effective in significantly reducing the incidence of ventricular arrhythmias and thus preventing sudden cardiac deaths in the mammalian SCD models.

Conventional techniques for studying arrhythmias, including ambulatory electrocardiography with computer-assisted analysis and programmed stimulation techniques for arrhythmia induction during intracardiac electrophysiological study, may be employed to determine the effectiveness of a specific dose or amount of a composition to may be used in treating arrhythmias. Other standard and well-known techniques may also be used in determining the effective amount of a particular agent to agonize β₃ adrenergic receptors.

The methods and embodiments illustrated with reference to the drawings and described herein are merely illustrative of the principles of the invention which may be implemented in, alternative embodiments to achieve other ends than those specifically described herein. Accordingly, the following examples are set forth for the purpose of illustration only and are not construed as limitations on the method disclosed herein.

### EXAMPLE 1

### Canine Model for Sudden Cardiac Death

An canine model for sudden cardiac death is disclosed in U.S. Pat. No. 6,351,668, which is incorporated herein by reference. The circumstances under which sudden cardiac death occurs in canine subjects are similar to circumstances under which sudden cardiac death occurs in human patients. Accordingly, a canine SCD model may be used to analyze and identify conditions within the heart leading up to a ventricular tachycardia or ventricular fibrillation of the type leading to sudden cardiac death, as disclosed in U.S. Pat. No. 6,353,757, which is incorporated herein by reference. A canine SCD model may also be used to develop and test the effectiveness of new techniques for preventing a ventricular tachycardia, ventricular fibrillation or sudden cardiac death from occurring, as disclosed in U.S. Pat. No. 6,398,800 and pending U.S. application Ser. No. 10/033,400, filed Dec. 12, 2001, which are incorporated herein by reference.

The canine SCD model is created by inducing myocardial hyperinnervation within the left stellate ganglion in combination with creating a complete atrioventricular (AV) block and inducing a relatively mild myocardial infarction (MI). The AV block is typically created by ablating the AV node of the heart using an ablation catheter and the MI is induced by ligating the left anterior descending portion of the coronary artery. Myocardial hyperinnervation is stimulated by application of nerve growth factor (NGF) or other neurtrophic vectors to the left stellate ganglion. Alternatively, electrical stimulation signals may be applied to the left stellate ganglion.

By creating an AV block and by inducing an MI within the heart of an adult canine test subject, and then by stimulating nerve growth within the left stellate ganglion of the subject using NGF, a significant increase in the likelihood of SCD arising from phase two ventricular arrhythmias has been observed. Thus, the method permits SCD to be induced within test animals in a manner facilitating the collection of data pertinent to conditions within the heart arising prior to SCD and for testing techniques intended to prevent phase two VT and VF within patients subject to a previous MI.

Four canine groups were evaluated in this study. Table 1 describes the treatment for each of the four canine groups. MI was created in each of the canine subjects by ligating the left anterior descending coronary artery. Complete AV block was created by radiofrequency ablation of the atrioventricular junction in the MI, SCD and RSG Groups. An ICD was used to monitor for the occurrence of ventricular arrhythmias. Nerve growth factor (NGF) was infused by an osmotic pump throughout a 4-5 week period in the left stellate ganglion (LSG) of the canine subjects in the SCD Group and in the right stellate ganglion (RSG) of the canine subjects in the RSG Group. The canine subjects in the SCD Group was characterized by a high incidence of SCD (44%), long QTc intervals and frequent episodes of ventricular tachycardia, whereas the RSG Group had no SCD, short QTc intervals and few episodes of VT.

TABLE 1
Canine Groups Used in the Study.

| Canine Groups | | Treatment |
|---|---|---|
| 1. | Control Group | No treatment |
| | (n = 6) | |
| 2. | MI Group | Myocardial infarction |
| | (n = 6) | Complete AV block |
| 3. | SCD Group | Myocardial infarction |
| | (n = 9) | Complete AV block |
| | | Nerve growth factor infusion to |
| | | the left stellate ganglion (LSG) |
| 4. | RSG Group | Myocardial infarction |
| | (n = 6) | Complete AV block |
| | | Nerve growth factor infusion to |
| | | the right stellate ganglion (RSG) |

### EXAMPLE 2

### Distribution Patterns and Co-Localization of β₁ and β₃ Adrenergic Receptors

The distribution pattern of β₁ and β₃ adrenergic receptors in the cardiac myocytes, Purkinje cells and small arteries and the partial co-localization of these receptors in the sarcolemma and Z bands of myocytes suggests possible interaction between β₁ and β₃ adrenergic receptors. The distribution pattern of β₁ and β₃ adrenergic receptors was demonstrated by immunohistochemical studies. Co-localization of these receptors were demonstrated by confocal microscopy.

Immunohistochemical studies. Fresh tissue specimens from the left ventricle around the posterior and anterior papillary muscles were routinely processed and embedded in paraffin. A modified immunohistochemical ABC method was used with the primary antibodies against β₁ adrenergic receptors (1:50 dilution, Santa Cruz Biotechnology) or β₃ adrenergic receptors (1:100, SmithKline Beecham). Parallel control experiments were conducted by using the blocking peptide for β₁ adrenergic receptor antibody or by omitting the primary β₃ adrenergic receptor antibody. The strength of the β₁ and β₃ adrenergic receptor positive staining was measured by computer-assisted color hue analyses (Image Pro Plus 4.0). The line profile was set across a transverse diameter of 10 myocardial cells and avoiding the nucleus. Color hue numbers were then evaluated and averaged. Positive stains resulted in a brown hue, which correlated with smaller hue values. Therefore, the hue number is inversely proportional to the strength of positive staining.

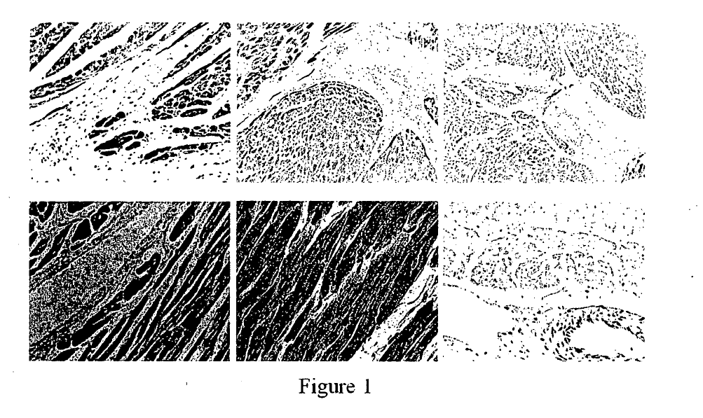

FIG. 1 depicts immunohistochemical staining of β₃ adrenergic receptors (FIGS. 1A-B), β₁ adrenergic receptors (FIGS. 1C-E), and a negative control (FIG. 1F). As shown in FIGS. 1A-B, the immunohistochemical staining localized β₃ adrenergic receptors primarily to cardiac myocytes, Purkinje fibers and small arteries. Arterioles were faintly stained for β₃ adrenergic receptors. No obvious staining of the nerves, veins or interstitial cells was observed.

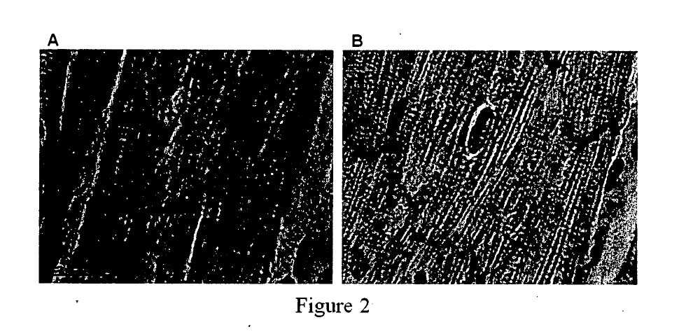

As shown in FIGS. 1C-E, the staining of β₁ adrenergic receptor showed similar patterns, with positive staining in cardiac myocytes, Purkinje cells and small arteries, and no obvious staining of the nerves, veins or interstitial cells. The negative control staining in FIG. 1F shows no detectable staining above the background when the primary antibody was omitted or when a blocking peptide was used. The immunohistochemical staining of β₃ and β₁ adrenergic receptors under 100× magnification, depicted in FIGS. 2A and 2B, respectively, shows the most abundant staining in the cell membrane and intercalated discs.

Double-immunofluorescence staining and confocal microscopy. Fresh tissue specimens from the left ventricle were harvested from the SCD Group, embedded in OCT standard solution and cut with a cryostat into 6μm thick tissue sections. The tissue sections were laid out on gelatin-coated slides, fixed in 4% formalin for 10 minutes, and then rinsed 3 times with PBS. The tissue sections were incubated with primary antibodies against β₁ adrenergic receptors (1:50) and β₃ adrenergic receptors (1:100) for one hour, followed by the application of Alexa Fluor 568 goat anti-rabbit IgG and Alexa Fluor 488 goat anti-rat IgG (1:800, Molecular Probes) for 2 hours in darkness. Stained sections were mounted in Immuno Floure Mounting Medium (ICN Biochemicals). Immunofluorescence was then detected by Leica TCS SP laser scanning confocal microscope with oil immersion lens.

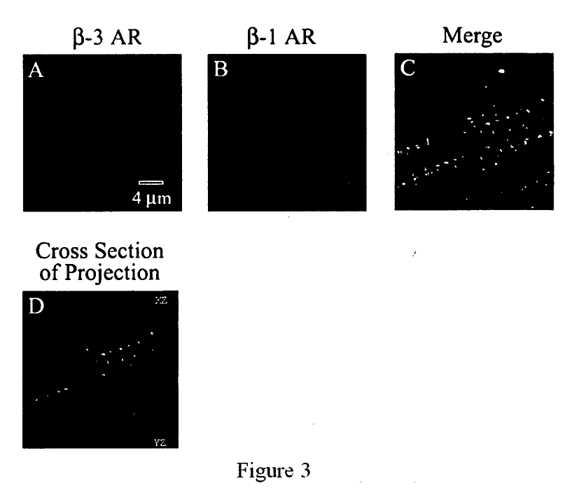

Confocal microscopic imaging of the double-immunofluorescence stained tissue sections was performed to determine whether β₁ and β₃ adrenergic receptors co-localized in the same sites. As shown in FIG. 3, intense fluorescent signals of β₁ and β₃ adrenergic receptor staining were largely concentrated in the sarcolemma and Z bands of myocytes, with little signal in the cytosol, showing a similar striated appearance but only co-localization. FIG. 3A shows positive β₃ adrenergic receptor staining in green within sarcolemma and Z bands within the cardiac myocytes and FIG. 3B shows positive β₁ adrenergic receptor staining in red, also within the sarcolemma and Z bands of cardiac myocytes. FIG. 3C is a merged view of FIGS. 3A and 3B showing co-localization (in yellow) of the β₁ and β₃ adrenergic receptors. FIG. 3D shows the image analysis of the cross section of the merged maximum intensity projection of FIG. 3C along the XZ and YZ planes of the three-dimensional volume of a stack of optical sections (0.5 μm thick) from a Z-series, starting from the crosshair marker. FIG. 3D clearly shows that the two receptors have inhomogeneous and distinct profiles of relative distribution in cardiac myocytes in situ.

### EXAMPLE 3

### Immunoreactivity of β Adrenergic Receptors in the Canine Groups

β₃ adrenergic receptor immunoreactivity increased in the SCD Group but decreased in the RSG Group. No significant change was observed in β₁ and β₂ adrenergic receptor protein levels in the SCD Group. Statistical analyses of color hue numbers from three separate staining sessions were performed. Table 2 provides the color hue numbers obtained for the β₁ and β₃ adrenergic receptors in the four canine groups.

In each session, β₃ adrenergic receptor staining showed the strongest signal in the SCD Group and the weakest signal in the RSG Group. The color hue number of β₃ adrenergic receptor staining in the SCD Group (42±16) was significantly lower (p<0.05 for both) than the color hue number of β₃ adrenergic receptor staining in the MI Group (60±9) and the Control Group (63±11). As previously indicated, the color hue number is inversely proportional to the staining strength. Accordingly, this indicates that the SCD Group had a higher β₃ adrenergic receptor immunoreactivity than both the MI and Control Groups.

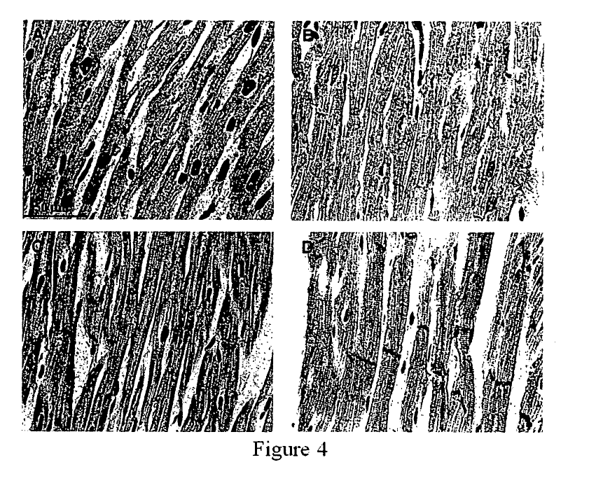

In contrast, the color hue of the β₃ adrenergic receptor staining in the RSG Group (92±9) was significantly higher than in other groups (p<0.01 for all), suggesting that the RSG Group had the lowest immunoreactivity. There was no significant difference in β₃ adrenergic receptor staining between the MI and Control Groups. FIG. 4 shows typical examples of β₃ adrenergic receptor staining in all four groups as follows: Control Group (FIG. 4A), MI Group (FIG. 4B), RSG Group (FIG. 4C), and SCD Group (FIG. 4D). Positive staining is indicated by brown color. FIG. 4D shows the strongest brown color, with positive staining appearing in the cell membrane and intercalated disks of the myocytes, while FIG. 4C shows the weakest brown color.

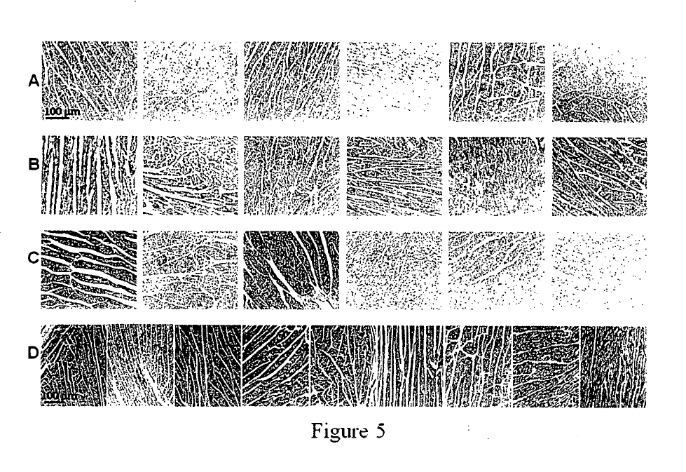

FIG. 5 shows examples of β₃ adrenergic receptor staining in all twenty-seven canine subjects studied from the Control Group (FIG. 5A), the MI Group (FIG. 5B), the RSG Group (FIG. 5C), and the SCD Group (FIG. 5D). The SCD Group had the strongest staining (most brown) and the RSG Group had the weakest staining (least brown). No significant differences were observed between the staining in the Control and MI Groups, as shown in FIGS. 5A and 5B.

While there were significant differences in β₃ adrenergic receptor staining, no significant differences in color hue numbers of β₁ adrenergic receptor staining was observed among the SCD Group (53±7), RSG Group (50±10), MI Group (56±10), and Control Group (59±12). These results suggest that there was no significant difference in β₁ adrenergic receptor immunoreactivity among these groups.

TABLE 2
β₁ and β₃ Color Hue Numbers for the Canine Groups.

| | | β₁ adrenergic | β₃ adrenergic |
|---|---|---|---|
| Canine Groups | | receptor | receptor |
| 1. | Control Group (n = 6) | 59 ± 12 | 63 ± 11 |
| 2. | MI Group (n = 6) | 56 ± 10 | 60 ± 9 |
| 3. | SCD Group (n = 9) | 53 ± 7 | 42 ± 16 |
| 4. | RSG Group (n = 6) | 50 ± 10 | 92 ± 9 |

### EXAMPLE 4

### Upregulation of β₃ Adrenergic Receptors in the Canine Sudden Cardiac Death Model

Fresh tissue samples from the non-infarcted left ventricular free wall using sharp skin biopsy punches (Acu-Punch, Acuderm, Inc.) were obtained for protein and mRNA analyses from canine subjects in the Control and SCD Groups. The excised tissues were immediately frozen in liquid nitrogen for further processing.

Western Blotting Studies. The tissue samples were homogenized on ice with a cell lysis buffer (Cell Signaling Technology) and supernatants were collected as total lysates. Equal amounts (60 μg) of denatured proteins were fractionated on 4-20% Gradient Minigel (CPL) and transferred to PVDF membranes (Bio-Rad). The PVDF membranes were blocked with 5% nonfat dry milk in PBST (containing 0.05% Tween 20) and then incubated overnight at 4° C. with the primary antibody (β₁ and β₂ adrenergic receptors, 1:200 for both (Santa Cruz Biotechnology); β₃ adrenergic receptors, 1:100; nerve growth factor, 1:200, Santa Cruz Biotechnology and tyrosine hydroxylase (TH), 1:500, Chemicon. The tissue samples were then washed in PBST, incubated with horseradish peroxidase-conjugated second antibody, and revealed by Immun-Star HRP Substrate (Bio-Rad). For normalization, the same Western blots were re-probed with anti-GAPDH at 1:10,000 dilution (Research Diagnostics, Inc.). The density of bands on Western blots was quantified through the use of Kodak imaging stations.

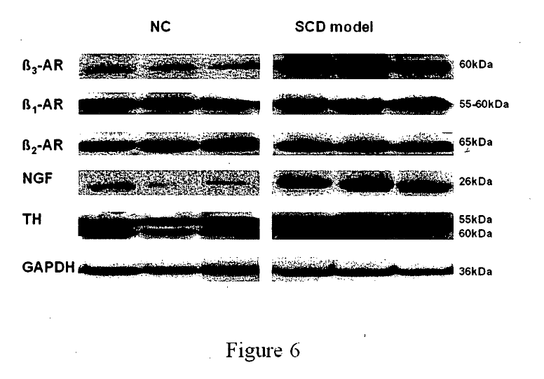

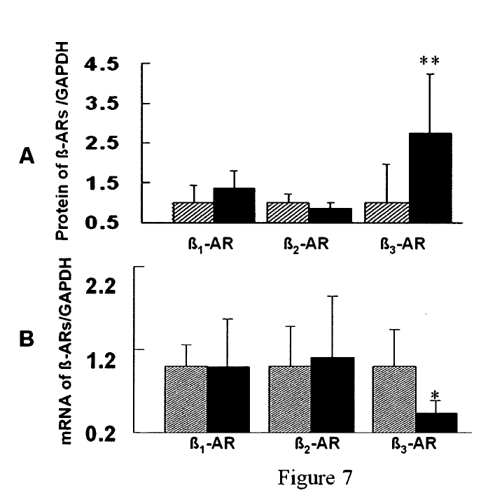

Upregulation of β₃ adrenergic receptor protein expression in the SCD model. The β₃ adrenergic receptor protein level was evaluated using Western blotting analysis. As shown in FIG. 6, a single band of β₃ adrenergic receptor was detected at around 60 kDa. The signal ratio of β₃ adrenergic receptor to GAPDH in the SCD Group (2.68±1.4) was significantly higher than that in the Control Group (1.0±0.9, P<0.001), indicating increased β₃ adrenergic receptor protein levels in the SCD Group (FIGS. 6 and 7A). FIG. 6 shows the representative Western blotting bands of β₃, β₁ and β₂ adrenergic receptors, NGF, TH, and GAPDH in the Control and the SCD Groups. The signal ratios of β₃ adrenergic receptor, NGF and TH to GAPDH were significantly increased in the SCD Group.

No change of β₁ or β₂ adrenergic receptor expression in the SCD model. As shown in FIG. 6, analysis of Western blots indicated no significant differences in β₁ adrenergic receptor protein levels in the SCD Group as compared to the Control Group (1.36±0.43 vs. 1.0±0.42, FIGS. 6 and 7A). FIG. 7 shows a graph representing the β adrenergic receptor protein expression level in the Control Group (hatched columns) and in the SCD Group (filled columns). Similarly, there were no significant differences in β₂ adrenergic receptor protein levels between the SCD and the Control Groups (0.85±0.12 vs. 1.0±0.2, FIGS. 6 and 7A). However, a significant increase in β₃ adrenergic receptor protein levels was observed in the SCD Group (**, p<0.01).

Increased NGF and TH proteins in the heart in the SCD model. It has previously been demonstrated that NGF infusion to the LSG in dogs with MI+complete AV block results in significant sympathetic hyperinnervation. Western blotting analysis further confirmed that NGF and TH protein levels were increased in the heart of the canine subjects in the SCD Group. As shown in FIG. 6, the NGF and TH bands were consistently denser and wider than the bands from the Control Group. Densitometric data showed that there was a significant increase in NGF protein level in the SCD Group than the Control Group (0.61±0.10 vs. 0.26±0.10, p<0.01). The TH protein level also higher in the SCD Group as compared to the Control Group (2.10±0.2 vs. 1.78±0.13, p<0.05). Moreover, a high correlation was found between the NGF and TH levels (R=0.78, p<0.01). This indicates that there was significant hyperinnervation resulting from high NGF level in the SCD Group.

mRNA Analyses. Total RNA was extracted using TRIZOL (Invitrogen) according to the manufacturer's protocol and treated with Dnase I (Qiagen) to degrade traces of DNA and cleaned with RNeasy Mini Kit (Qiagen). The concentration of RNA was quantified by determination of optical density at 260 nm (OD₂₆₀) and the integrity of each sample was confirmed by analysis on an agarose gel. Total RNA was reversely transcripted with TaqMan Reverse Transcription Reagents (Applied Biosystems).

The expression levels of candidate genes were measured by quantitative real-time RT-PCR (qRT-PCR) using SYBR Green PCR Master mix (Applied Biosystems) and canine specific primers on an ABI PRISM 7700 Sequence Detection System (Applied Biosystems) according to the manufacturer's protocol. The cycle at which amplification plot crosses the threshold is known to accurately reflect relative mRNA expression levels.

In each assay, both GAPDH (an endogenous control) and target genes from the same samples were amplified in duplicate separate tubes. Levels of mRNA of each gene were calculated using the relative standard curve method and normalized against corresponding GAPDH mRNA levels, then expressed in a relationship of relative change over control. A single dissociation peak was confirmed in each reaction by dissociation curve. The size of expected amplicons were confirmed by gel electrophoreses. The sequences of the genes studied were obtained from GenBank and the primers were designed using the Primer Express™ software (Applied Biosystems). Table 3 shows the primer sequences and amplicon size of the selected genes.

TABLE 3

Primer Sequence and Amplicon Size of Genes
Validated by Taqman RT-PCR

| | | | Amplicon |
|---|---|---|---|
| Gene Name | Accession No. | Primer Sequence | Size (bp) |
| β₁ adrenergic | U73207 | F: 5′-CATCATCATGGGCGTGTTCA-3′ | 57 |
| receptor | | R: 5′-ACCACGTTGGCCAGGAAGA-3′ | |
| β₂ adrenergic | X94608 | F: 5′-TGCCTTCCAGGAGCTTCTGT-3′ | 51 |
| receptor | | R: 5′-CCATAGGCCTTCAGGGAAGAC-3′ | |
| β₃ adrenergic | U92468 | F: 5′-GGGTTCTGTCCCTGACTCCAT-3′ | 51 |
| receptor | | R: 5′-GGGAAGGCTGGTGCTTAGGA-3′ | |
| GAPDH | AB038240 | F: 5′-AAAGCTGCCAAATATGACGACAT-3′ | 51 |
| | | R: 5′-CTCCGATGCCTGCTTCACTAC-3′ | |

β₃ adrenergic receptor mRNA expression was assessed by use of qRT-PCR. FIG. 7B shows the β adrenergic receptor mRNA levels in the Control and SCD Groups. Similar to the protein expression patterns, there were no significant differences in the β₁ and β₂ adrenergic receptor mRNA expression between the Control and SCD Groups. However, the β₃ adrenergic receptor mRNA level was much lower in the SCD Group than in the Control Group. The value of the signal ratio of β₃ adrenergic receptor to GAPDH in the SCD Group (0.43±0.15) was lower than that in the Control Group (1.0±0.44, p<0.05), as shown in FIG. 7B. This suggests decreased β₃ adrenergic receptor mRNA expression. he mRNA levels for β₁ adrenergic receptor in the SCD Group (0.99±0.57) and in the Control Group: 1.0±0.26) were not significantly different. Similarly, the mRNA levels for β₂ adrenergic receptor receptors in the SCD Group (1.1±0.7) and the Control Group (1.0±0.48) was not significantly different.

### EXAMPLE 5

### Upregulation of β₃ Adrenergic Receptors in the Canine Acute Myocardial Infarction Model

The temporal variation of β₃ adrenergic receptor expression after myocardial infarction (MI) was also investigated by analyzing β₃ adrenergic receptor mRNA and protein levels at the non-infarcted left ventricular free wall at 3.5 hours, 3 days, 1 week and 1 month after MI using qRT-PCR and Western blotting. MI was created in the canine subjects by occluding the left anterior descending coronary artery. Samples from the non-infarcted left ventricular free wall were harvested at 3.5 hours (n=3), 3 days (n=5), 1 week (n=5) and 1 month (n=3) after MI. Six canine subjects were used as control. mRNA and protein levels of β₁, β₂ and β₃ adrenergic receptors were studied through the use of qRT-PCR and Western blotting.

At 3.5 hours after MI, the β₁ adrenergic receptor mRNA level in the MI Group was not significantly different from the Control Group (1.02±0.25 vs. 1.0±0.24). At 3 days after MI, the MI Group showed a significant increase in β₃ adrenergic receptor mRNA level compared to the Control Group (3.4±1.6 vs. 1.0±0.24, p<0.01). At 1 week after MI, β₃ adrenergic receptor mRNA level in the MI Group increased by 59% (1.59±0.12 vs. 1.0±0.24, p<0.01). At 1 month after MI, no significant change in the β₃ adrenergic receptor expression was observed between the MI and Control Groups (p=NS).

Densitometric data of Western blot showed that the β₃ adrenergic receptor protein level was significantly higher in the MI Group than the Control Group at 3 days (1.42±0.24 vs. 1.0±0.34) and 1 week (1.53±0.21 vs. 1.0±0.34) after MI (p<0.05 for both). However, there were no significant changes at 3.5 hours and 1 month after MI.

Dynamic expression of β₁ and β₂ adrenergic receptor mRNA levels were observed after MI. β₁ adrenergic receptor mRNA level in MI Group showed significant increase at 3.5 hours after MI (1.68±0.21 vs. 1.0±0.26, p<0.01). β₂ adrenergic receptor mRNA level increased significantly at 1 week after MI (1.92±0.42 vs. 1.0±0.40, p<0.05). No significant changes were observed at any other time points.

Western blotting analysis showed that β₁ adrenergic receptor protein level increased by 72% at 3 days after MI (1.72±0.25 vs. 1.0±0.28, p<0.01), β₂ adrenergic receptor protein level increased at 3.5 hours after MI (1.42±0.20 vs. 1.0±0.18, p<0.05). There were no significant differences at any other time points.

### EXAMPLE 6

### Effects of Stimulating β₃ Adrenergic Receptors on SCD Models

The administration of β₃ adrenergic receptor agonist has been found to inhibit the occurrence of phase-2 ventricular tachycardia (“VT”) in subjects susceptible to SCD. Two canine groups were studied and compared to ascertain the effect of a β₃ adrenergic receptor agonist (BRL 37344) on the incidence of phase-2 VT, typically leading to sudden cardiac death. Table 4 compares the incidence of phase-2 VT, QTc intervals and the size of myocardial infarction (“MI”) of SCD canine subjects and SCD canine subjects receiving BRL 37344 infusion.

β₃ adrenergic receptor agonist BRL 37344 was infused subcutaneously by an osmotic pump (2 μg/kg/hr) for two weeks in two canine subjects with myocardial infarction, complete atrioventricular block and nerve growth factor to the left stellate ganglion (BRL Group). Surface ECG was continuously recorded by Data Sciences International (DSI) and analyzed manually.

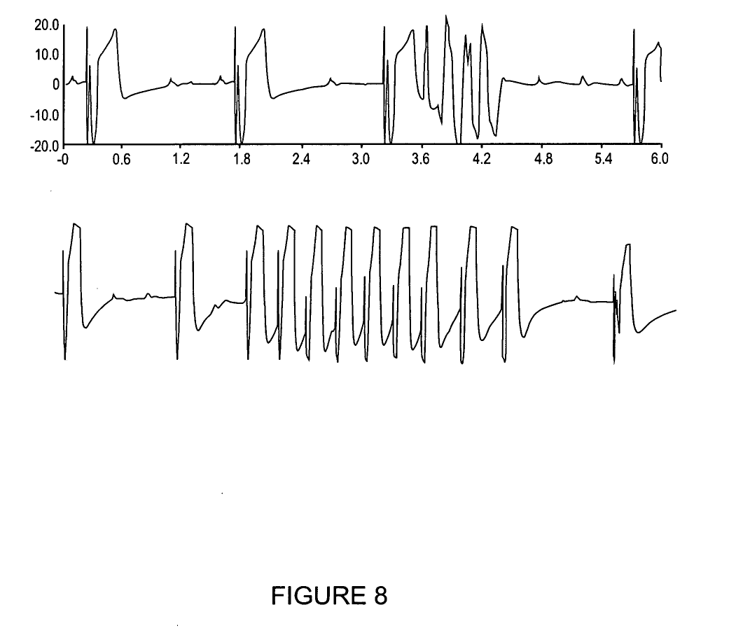

The two canine subjects in the BRL Group were followed up for two months without SCD. One canine subject had no phase-2 VT for five weeks and short runs ventricular tachycardia episodes were observed from day 36 to day 44 after surgery. The second canine subject had no phase-2 VT episodes. The incidence of phase-2 VT in the two canine subjects in the BRL Group (0.08±0.11 per day) was significantly less frequent than compared to that of the canine subjects in the SCD Group that did not receive BRL 37344 infusion (2.0±2.0 per day). FIG. 8 shows the ECG recordings of one of the canine subjects following surgery.

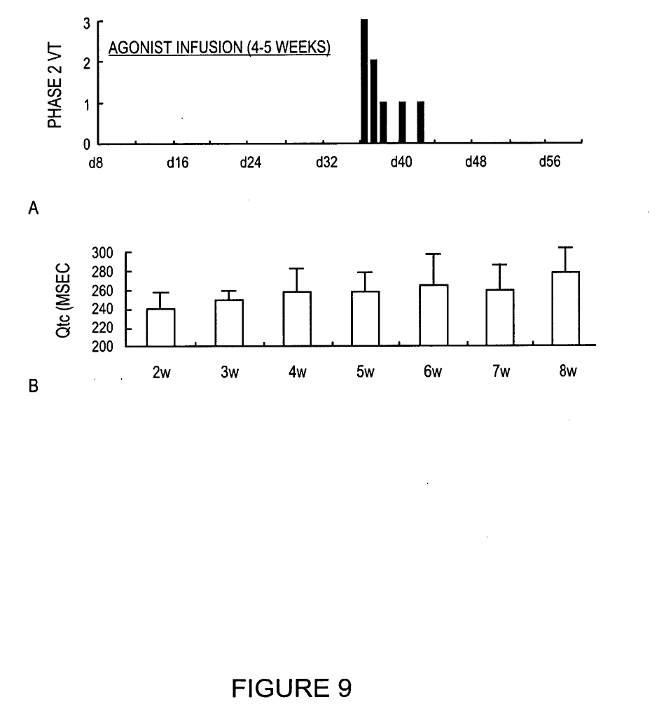

In addition, the QTc interval was shorter throughout follow-up for the canine subjects in the SCD Group receiving BRL 37344 (350 ms) as compared to the canine subjects in the SCD Group that did not receive BRL 37344 (400 ms). The myocardial infarction size of the canine subjects in the SCD Group receiving BRL 37344 (16±1.2%) is similar to SCD Group that did not receive BRL 37344 infusion (17±4%). FIG. 9A shows the occurrence of phase 2 VT episodes in the two dogs and FIG. 9B is a graph showing the QTc intervals of the two dogs throughout follow-up.

TABLE 4
Comparison of SCD Canine Models to SCD Canine
Models Receiving BRL 37344 Infusion

| Canine Group | Incidence of Phase-2 VT | QTc Interval | Size of MI |
|---|---|---|---|
| SCD | 2.0 ± 2.0 per day | 400 ms | 17 ± 4% |
| SCD + BRL | 0.08 ± 0.11 per day | 350 ms | 16 ± 1.2% |
| 37344 infusion | | | |

What is claimed:

1. A method for reducing the likelihood of the occurrence of a cardiac arrhythmia in a mammalian subject, the method comprising administering to the mammalian subject an effective amount of a composition to agonize β₃ adrenergic receptors in the mammalian subject.

2. The method of claim 1 wherein the composition comprises at least one agent selected from the group consisting of: BRL 37344, CPG 12177, CL 316243, SR 58661, pindolol, and cyanopindolol.

3. The method of claim 2 wherein the agent is BRL 37344.

4. The method of claim 2 wherein the composition further comprises agents that antagonize β₁ and/or β₂ adrenergic receptors.

5. The method of claim 3 wherein composition is administered intravenously.

6. The method of claim 3 wherein the composition is administered orally.


SEQUENCE LISTING (8 sequences)

SEQ ID NO: 1 - DNA, 20 bp, Canine
catcatcatgggcgtgttca

SEQ ID NO: 2 - DNA, 19 bp, Canine
accacgttggccaggaaga

SEQ ID NO: 3 - DNA, 20 bp, Canine
tgccttccaggagcttctgt

SEQ ID NO: 4 - DNA, 21 bp, Canine
ccataggccttcagggaagac

SEQ ID NO: 5 - DNA, 21 bp, Canine
gggttctgtccctgactccat

SEQ ID NO: 6 - DNA, 20 bp, Canine
gggaaggctggtgcttagga

SEQ ID NO: 7 - DNA, 23 bp, Canine
aaagctgccaaatatgacgacat

SEQ ID NO: 8 - DNA, 21 bp, Canine
ctccgatgcctgcttcactac